(19) United States
(12) Patent Application Publication
(10) Pub. No.: US 2020/0411200 A1
(43) Pub. Date: Dec. 31, 2020

(54) INFORMATION PROCESSING APPARATUS AND NON-TRANSITORY COMPUTER READABLE MEDIUM

(71) Applicant: FUJI XEROX CO., LTD., Tokyo (JP)

(72) Inventor: Morio OKA, Kanagawa (JP)

(73) Assignee: FUJI XEROX CO., LTD., Tokyo (JP)

(21) Appl. No.: 16/587,532

(22) Filed: Sep. 30, 2019

(30) Foreign Application Priority Data
Jun. 25, 2019 (JP) 2019-116851

(51) Int. Cl. G16H 70/40; G16H 50/20; G16H 10/60; G16H 20/10

(52) U.S. Cl. CPC G16H 70/40; G16H 20/10; G16H 10/60; G16H 50/20

(57) ABSTRACT

An information processing apparatus includes an extraction unit that extracts, from each sentence included in a document, medicine information identifying a medicine, symptom information related to a symptom, and change information representing a change in a condition of a patient, and a generation unit that, after the medicine is taken by the patient, generates structured information that is used to determine a side effect from the information extracted by the extraction unit.

### CROSS-REFERENCE TO RELATED APPLICATIONS

This application is based on and claims priority under 35 USC 119 from Japanese Patent Application No. 2019-116851 filed Jun. 25, 2019.

### BACKGROUND

### (i) Technical Field

The present disclosure relates to an information processing apparatus and a non-transitory computer readable medium.

### (ii) Related Art

Medicines may be used in medical care after they are tested in clinical trials and approved. In the clinical trials, a variety of examinations are performed on humans and information on the effect and safety of the medicines is collected. Even after one medicine is approved as a new medicine, information on side effect may be continuously collected for evaluation. If a case of side effect has occurred and information on that event has been reported by patients suffering from a variety of diseases, doctors or nurses, seriousness and a cause and effect relationship between medicine and symptom are evaluated from the information. If the side effect is serious (namely, the symptom is server), a safety job, such as reporting to regulatory authorities, may be performed.

Related art techniques are disclosed in Japanese Unexamined Patent Application Publication Nos. 2012-203772, 2017-211772, and 2017-199351.

One of the safety jobs includes a screening job that extracts information from documents, such as medical papers, and determines whether a symptom appearing after the use of a medicine corresponds to a side effect. The screening job includes extracting, from medical papers, information (namely, description) based on which the determination of the side effect has been performed. A job of extracting the information, namely, a job of extracting a description considered to be related to the side effect (also referred to as a marking job) is manually performed in the related art. Since the information simply manually extracted is not structured, it is difficult to handle it. Manually structuring of information involves workload and there is a possibility that the information serving as a basis of the determination of the side effect escapes detection.

### SUMMARY

Aspects of non-limiting embodiments of the present disclosure relate to enabling a side effect of a medicine to be more efficiently determined than when information related to side effect of a medicine described in a document is manually structured.

Aspects of certain non-limiting embodiments of the present disclosure address the above advantages and/or other advantages not described above. However, aspects of the non-limiting embodiments are not required to address the advantages described above, and aspects of the non-limiting embodiments of the present disclosure may not address advantages described above.

According to an aspect of the present disclosure, there is provided an information processing apparatus. The information processing apparatus includes an extraction unit that extracts, from each sentence included in a document, medicine information identifying a medicine, symptom information related to a symptom, and change information representing a change in a condition of a patient, and a generation unit that, after the medicine is taken by the patient, generates structured information that is used to determine a side effect from the information extracted by the extraction unit.

### BRIEF DESCRIPTION OF THE DRAWINGS

Exemplary embodiment of the present disclosure will be described in detail based on the following figures, wherein:

FIG. 1 is a block diagram illustrating an information processing apparatus of an exemplary embodiment;

FIG. 2 is a flowchart illustrating a side-effect determination process of the exemplary embodiment;

FIG. 3 specifically illustrates the side-effect determination process of the exemplary embodiment;

FIG. 4 illustrates a data structure of a structured table of the exemplary embodiment;

FIG. 5 illustrates a display example of a standard screen provided by the information processing apparatus of the exemplary embodiment;

FIG. 6 illustrates a display example of a document selection screen displayed when a side-effect extraction button is selected on the standard screen of the exemplary embodiment;

FIG. 7 illustrates a display example of a side-effect sub-screen displayed when a side-effect present button is selected on the standard screen of the exemplary embodiment;

FIG. 8 illustrates a display example of a side-effect sub-screen displayed when a side-effect absent button is selected on the standard screen of the exemplary embodiment;

FIG. 9 illustrates a display example of a document display screen displayed when a document highlight button is selected on the side-effect sub-screen of the exemplary embodiment;

FIG. 10 illustrates a document selection screen displayed when a document information extraction button is selected on the standard screen of the exemplary embodiment; and

FIG. 11 illustrates a display example of the document information display screen displayed when the document information display button is selected on the standard screen of the exemplary embodiment.

### DETAILED DESCRIPTION

Exemplary embodiment of the disclosure is described below with reference to the drawings.

An information processing apparatus 1 of the exemplary embodiment is implemented by a versatile hardware configuration of related art, such as a personal computer (PC). The information processing apparatus 1 of the exemplary embodiment thus includes a central processing unit (CPU), memories, such as a read-only memory (ROM), random-access memory (RAM), and hard disk drive (HDD), and communication media, such as a user interface and a network interface. The user interface may include a mouse and a keyboard, as an input unit, and a display. The user interface may include a touch panel liquid-crystal panel, serving as an input unit and a display unit.

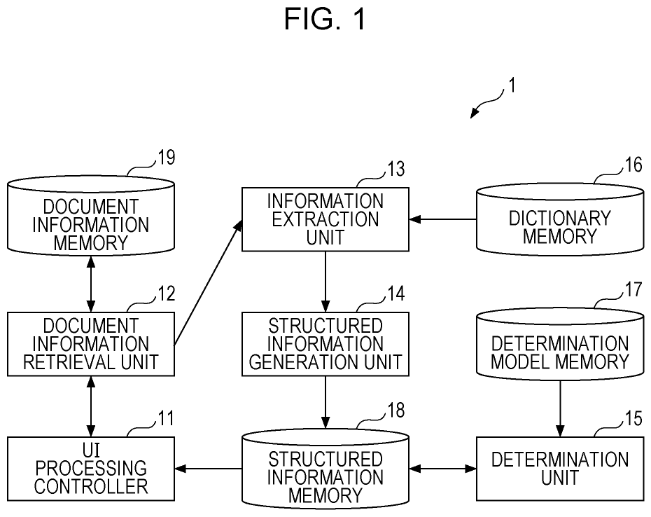

FIG. 1 is a block diagram illustrating the information processing apparatus 1 of the exemplary embodiment. The information processing apparatus 1 of the exemplary embodiment includes a user interface (UI) processing controller 11, document information acquisition unit 12, information extraction unit 13, structured-information generation unit 14, determination unit 15, dictionary memory 16, determination model memory 17, structured-information memory 18, and document information memory 19. Elements of the information processing apparatus 1 not described with reference to the exemplary embodiment are not illustrated in the drawings.

The UI processing controller 11 has a function of a display controller that performs control to display a variety of images on a display unit and a function of receiving unit that receives information that is entered on a display screen by a user who uses an input unit. The document information acquisition unit 12 retrieves, from an internal memory (not illustrated) or via a network, documents, such as medical papers, specified by a user as a determination target about side effect and including safety information. At least a document retrieved via the network is stored on the document information memory 19.

The information extraction unit 13 extracts as information a description considered to describe about a side effect from the document retrieved by the document information retrieval unit 12. The information extraction unit 13 of the exemplary embodiment extracts, from each sentence included in the document, medical information identifying a medicine described in the document, symptom information on a symptom, and change information representing a change in a condition of a patient. When the information is extracted, it is still unclear whether or not the information is related to a side effect. This determination is performed based on determination results provided by the determination unit 15.

The structured-information generation unit 14 generates structured information and stores it on the structured-information memory 18. The structured information is extracted by the information extraction unit 13 on a per sentence basis and includes the medicine information, symptom information, and change information. The structured information is used to determine a side effect when the medicine identified by the medicine information is taken by a patient. The determination unit 15 analyzes the generated structured information not only on the document but also on each sentence and determines the side effect of the medicine that is written in the document. Specifically, the determination unit 15 determines the side effect on a per sentence basis if the medicine identified by the medicine information included in the structured information of each sentence is taken by the patient. The determination unit 15 thus additionally registers on the structured-information memory 18 determination results of the side effect with respect to each sentence in association with the side effect of the sentence. The determination unit 15 of the exemplary embodiment may determine the side effect by using a learning model produced through machine learning. In accordance with the exemplary embodiment, the determination of the side effect using the learning model is also referred to as an artificial intelligence (AI) determination.

The dictionary memory 16 stores multiple dictionaries used by the information extraction unit 13. For example, the information extraction unit 13 stores side-effect dictionaries, including a medicinal dictionary referred to when the information extraction unit 13 refers to medicine names, an all-disease dictionary referred to when symptom, and disease name are extracted, and medical dictionary for regulatory activities (MedDRA) referred to when side effects are searched. The dictionary memory 16 further stores a specified dictionary related to medical care, such as a medical jargon dictionary. The dictionary memory 16 further stores a dictionary of synonyms that is used to accounting for word variations. The types of dictionaries are quoted here for exemplary purposes only and the disclosure is not limited to these dictionaries.

As described above, the determination model memory 17 stores the learning model (hereinafter referred to as “determination model”) used when the determination unit 15 performs the AI determination and the structured-information memory 18 stores the structured information generated by the structured-information generation unit 14. The document information memory 19 stores document data and literature information of the document that the user has specified as a side-effect determination target.

The elements of the information processing apparatus 1, namely, the UI processing controller 11 through the determination unit 15, are implemented when a computer forming the information processing apparatus 1 and a program running on the CPU on the computer operate in concert with each other. The dictionary memory 16 through the document information memory 19 are implemented by a hard disk drive (HDD) on the information processing apparatus 1. Alternatively, the RAM may be used or a memory external to the information processing apparatus 1 may be used via the network.

The program used in the exemplary embodiment may be provided via a communication medium. The program may also be provided in a recorded form on one of non-transitory computer-readable recording media, including a compact-disk read-only memory (CD-ROM) and universal serial bus (USB) memory. The program provided via the communication medium or the recording medium is installed on the computer and the CPU in the computer successively performs the program. A variety of processes are thus executed.

In accordance with the exemplary embodiment, the side effect of each medicine is determined by automatically extracting, from a document referred to in the determination of a side effect of a medicine (medicine side effect), information considered to include a description of the side effect, and by structuring the extracted information, and by analyzing the structured information. When the side effect of the medicine is determined, AI is used. To use AI, the determination model is produced in advance. The determination model is produced as described below.

A document that is determined to include a description of the medicine side effect and a document that is determined to include no description of the medicine side effect are prepared in advance as teacher data. The determination model is produced by entering the teacher data thereto. Since the documents serving as the teacher data may possibly include technical terms related to medical care and word variations, a variety of dictionaries stored on the dictionary memory 16 are desirably referred to. The determination model is produced in advance and a learning process is desirably repeated to improve the determination accuracy of the determination of the side effect.

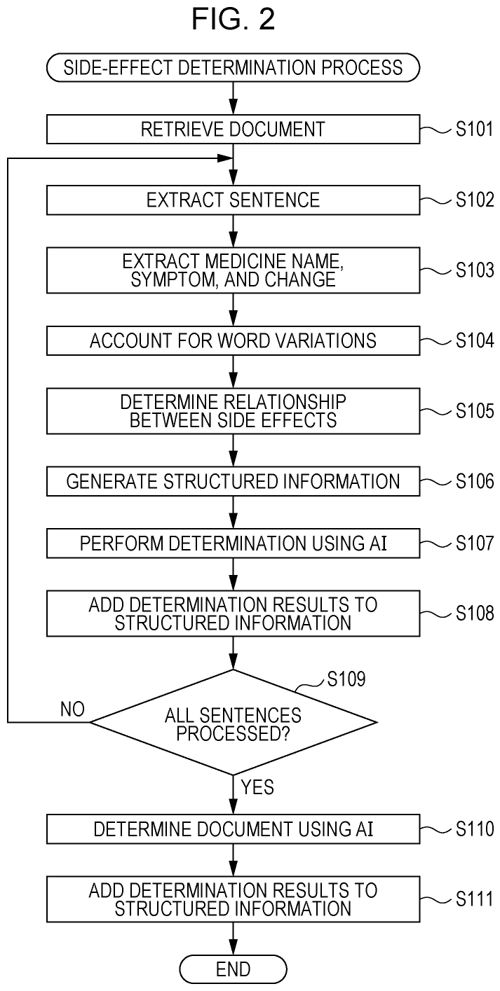

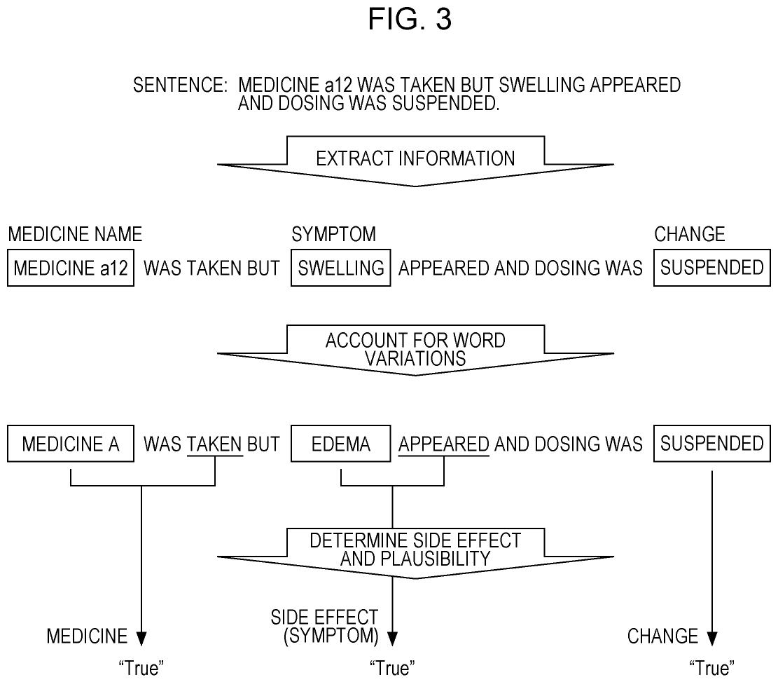

The process of the exemplary embodiment is described below. In accordance with the exemplary embodiment, the medicine side effect is determined by analyzing the document. FIG. 2 is a flowchart illustrating a side-effect determination process of the exemplary embodiment. FIG. 3 specifically illustrates a specific example of the side-effect determination process of the exemplary embodiment. With reference to FIGS. 2 and 3, the side-effect determination process is described below.

The document serving as the side-effect determination target includes multiple sentences regardless of whether a description related to a side effect is present in the document. If information identifying a document serving as a side-effect determination target, such as a document name, is specified by a predetermined operation of the user, the UI processing controller 11 receives the document name. A user interface that is used for the user to specify the document name and is used to display the information in response to the user operation is described later. The document information retrieval unit 12 retrieves the document corresponding to the document name specified by the user from a storage destination specified together with the document name (step S101).

The information extraction unit 13 executes the process described below on each sentence included in the document retrieved by the document information retrieval unit 12. To this end, the information extraction unit 13 extracts one sentence unprocessed from the document (step S102). The order of sentence extraction is not limited to any particular order. The sentences may be simply extracted in the order of appearance from the top of the document first.

The information extraction unit 13 analyzes the extracted sentence while referring to each of the dictionaries registered on the dictionary memory 16 and. The information extraction unit 13 thus extracts, as the medicine information, the symptom information, and the change information, the words corresponding to the medicine name, symptom, and change included in the sentence (step S103). Referring to FIG. 3, a sentence reading “Medicine a12 was taken but swelling was observed and dosing was suspended” is extracted. From these sentences, words “medicine a12” are extracted as a medicine name, the word “swelling” is extracted as a symptom, and the word “suspended” is extracted as a change.

The “medicine name” is the name of a medicine that is registered in a medicinal dictionary. The “symptom” refers to a disease or the condition of the disease. In accordance with the exemplary embodiment, the symptom has a broad sense. A disease registered in a side-effect dictionary or the name of a disease itself (namely, a disease name) may be extracted as the symptom. The “change” refers to a change in the symptom of a patient. The information is basically words registered in the medical jargon dictionary. The information is not limited to the words. The past extracted results of the change information may be stored as a database and the information may be extracted by referring to the database.

The words corresponding to the medicine name, symptom, and change are thus extracted. The information extraction unit 13 refers to the specified dictionary and the dictionary of synonyms and modifies the extracted words to account for the word variations (step S104). FIG. 3 illustrates an example of accounting for the word variations in which “medicine a12” is modified to “medicine A” and “swelling” is modified to “edema”.

In step S103, the words corresponding to the medicine name, symptom, and change are simply extracted and it is still unclear how each word is used in each sentence. For example, with “medicine a12” alone, it is unclear whether the medicine has been really used. If the medicine has not been used, a description of “medicine a12” does not lead to the determination that a side effect has been observed.

The information extraction unit 13 performs natural language processing, such as morphological analysis, on the process target to determine whether the medicine has been used. Terms indicating the use of medicine include “use”, “dose”, and other words. A determination as to whether a word corresponds to a word meaning use may be made by referring to the medical jargon dictionary and the past use results of words. In accordance with the exemplary embodiment, the expression meaning the use of medicine is generally expressed by “use”. Since the use of medicine is confirmed by the sentence “medicine a12 was taken” in FIG. 3, there is a possibility of a side effect. The information extraction unit 13 thus determines the “medicine” identified by the “medicine name” has a relationship with the “use”. In accordance with the exemplary embodiment, the result determined as having a relationship is indicated by the word “true” and the result determined as having no relationship is indicated by the word “false”.

Similarly, the word “edema” alone does not clarify whether or not edema has been observed. The information extraction unit 13 thus performs natural language processing, such as morphological analysis, on the process target sentence to determine whether edema has actually been observed. The detection of the symptom may be expressed by words “appearance” and “onset”. In the same way as with the word “use”, the determination is made by referring to the medical jargon dictionary. In accordance with the exemplary embodiment, the general expression intended to mean that the symptom appears is generally referred to as “appearance”. The sentence “swelling appeared” in FIG. 3 means that the appearance of edema is thus recognized and there is a possibility of a side effect. The information extraction unit 13 thus determines that a relationship between “symptom” and “appearance” is true. If a symptom without using medicine A (such as headache) appears, there is no relationship between the medicine A and the symptom. Since there is no relationship between the medicine A and the side effect, the information extraction unit 13 determines from the sentence that the relationship is “false”.

The word “suspend” alone does not clarify whether or not the dosing has been actually suspended. Natural language process is thus performed to determine whether the dosing has been actually suspended. For the word “change”, if a change has “occurred”, the relationship is true, and if a change has “not occurred”, the relationship is false. Since the sentence in FIG. 3 indicates that the word “suspended” defines the suspense of the dosing, there is a possibility of side effect. The information extraction unit 13 thus determines that the relationship between the word “change” and the word “occurred” is true.

Words corresponding to the medicine name and symptom are not so difficult to extract if an academic dedicated dictionary is referred to. Words indicating change are not limited to technical terms. A dictionary generated via AI by inputting the past results of text structural analysis is registered on the dictionary memory 16 and the words corresponding to the word “change” may be extracted. In this way, the word change is appropriately extracted.

As previously described, the determination of the side effect of a medicine involves the fact that the medicine has been actually used. Also, the determination of the side effect of a medicine involves a change that has been triggered by the use of the medicine. The information extraction unit 13 extracts information about a medicine, symptom, and change such that the information clarifies the fact about the symptom and change that have been triggered by the use of the medicine.

When the information extraction unit 13 extracts the information, the structured-information generation unit 14 generates the structured information by aggregating the extracted information and registers the structured information on the structured-information memory 18 (step S106). The structured information includes information on the presence or absence of relationship between medicine names, symptoms, and changes. Regardless of whether the relationship is true or false, the structured-information generation unit 14 generates and registers the structured information on the structured-information memory 18.

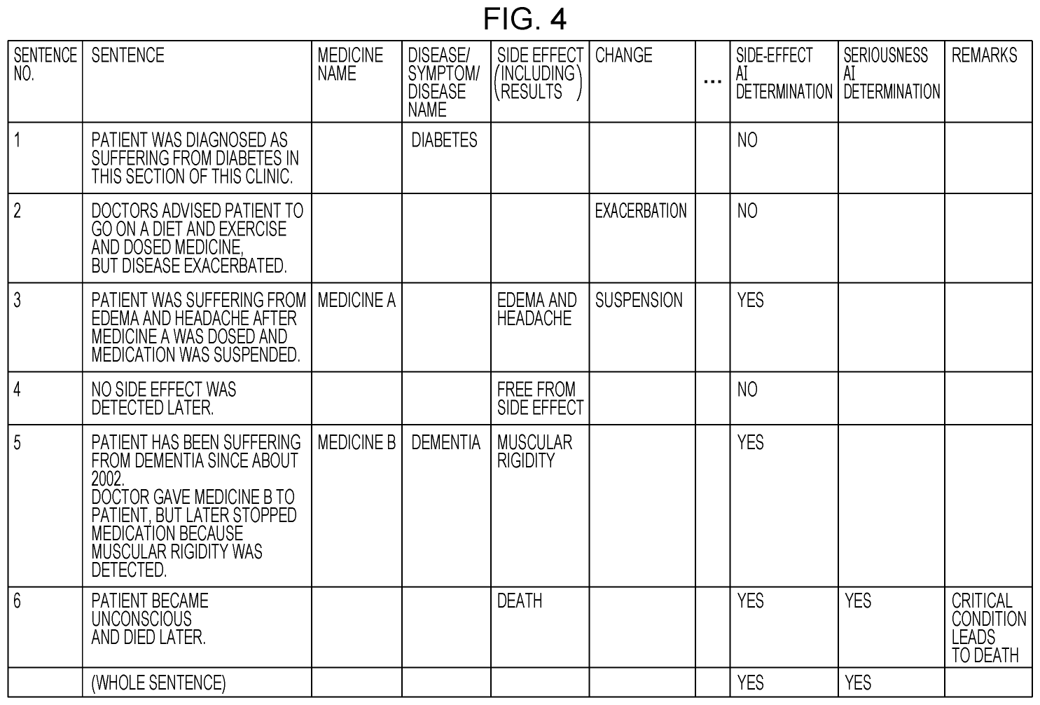

FIG. 4 illustrates a data structure of a structured table of the exemplary embodiment. In accordance with the exemplary embodiment, the structured information is managed in the form of table as illustrated in FIG. 4 and is then displayed to the user. The structured information in the form of table is hereinafter referred to as a “structured table”.

The structured information is generated on a per sentence basis. The structured information in FIG. 4 illustrates a sentence number respectively attached to a sentence serving as an information generation target and the structured information associated with the sentence. Medicine names included in the structured information are those that are extracted from the corresponding sentences by the information extraction unit 13. Symptoms extracted by the information extraction unit 13 are entered into a disease/symptom/disease name item column or a side-effect (including results) column. Changes extracted by the information extraction unit 13 are entered into the side-effect (including results) item column or change item column.

For example, diabetes is not a side effect but a disease name. Allergy is a disease and may also be a side effect. Depending on the context of the sentence, the item column to which the word is to be entered becomes different. For this reason, the structured information generated by the structured-information generation unit 14 is not necessarily correct. The user may thus be allowed to correct the structured table or the structured-information generation unit 14 may have the function of additionally registering an information item. Items for the AI determination included in the structured table may be described later.

When the structured information is generated, the determination unit 15 refers to the relationship of the context, in particular, words (namely, a medicine name, symptom, and change) and determines a side effect from the sentence by using a determination model (step S107). The relationship between the fact of using a medicine and the appearance of a side effect may be considered first and a change may be information to be considered next. Note that the determination unit 15 refers to the change to finally determine whether a side effect has appeared. For example, if any change has occurred, there is a higher possibility that the side effect is caused. In accordance with the exemplary embodiment, a change is input to the determination model as a feature quantity related to the side effect.

In accordance with the exemplary embodiment, the expression “determining the side effect” includes not only determining whether or not a medicine has been used and determining the presence or absence of a side effect, based on the fact, such as the occurrence of the symptom, but also determining the level of the side effect, such as the seriousness of the side effect. Newness of the side effect may also be considered although such a factor is not listed in FIG. 4. The newness of the side effect means that there is no description of any side effect of a medicine in a document attached to the medicine. The newness is divided into a known effect and an unknown effect. The known effect refers to a side effect that is described in the document attached to the medicine. On the other hand, the unknown side effect refers to a side effect that is not described in the attached document.

FIG. 4 lists two types of determination, namely, “side-effect AI determination” indicating the presence or absence of side effect and “seriousness AI determination” indicating whether the side effect is serious or not. This is related to the side effect of the medicine. If there is no description about any medicine name in the sentence, the medicine serving as a determination target with respect to the side effect is not known and the determination unit 15 determines that there is no side effect. If there is a description of a medicine name in the sentence, the determination unit 15 performs the AI determination with respect to the side effect of the medicine by inputting the medicine name, symptom, and change and relationship information to the determination model.

The “true” information and “false” information representing the presence or absence of the relationship are included in the structured information but are not included in the structured table displayed to the user. The presence or absence of the relationship may be included in the structured table. If the presence or absence of the relationship is included the structured table, the whole table displayed is difficult to view. The structured table is thus displayed without the presence or absence of the relationship. However, the determination unit 15 refers to the information indicating the presence or absence of the relationship when the side effect is determined. If the results of the AI determination are denoted by yes, the relationship between the medicine and the disease is presumed to be present.

The determination unit 15 performs the AI determination in this way and additionally registers the determination results onto the structured information that the information extraction unit 13 has generated in response to the sentence as the process target (step S108).

The process described above is repeated on each of the sentences included in the document (no path in step S109). When the process is complete on all the sentences in the document (yes path in step S109), the determination unit 15 determines side effect for the document (step S110). Specifically, if the side-effect AI determination is performed on a sentence and the number of results indicating a side effect is one, the determination unit 15 determines that a side effect is observed for that sentence. If the number of results indicating a side effect is zero, the determination unit 15 determines that no side effect is observed for that sentence.

As described above, in accordance with the exemplary embodiment, the side-effect determination process is performed on each sentence. Alternatively, the side-effect determination process may be performed on only a per document basis. In that case, however, the user may not be able to recognize the reason why the side effect has been observed from the document. In accordance with the exemplary embodiment, when the side-effect determination process is made on each sentence and the side-effect determination results are thus displayed together with the medicine information, symptom information, and change information. The user may thus be able to recognize the reason why the side effect has been observed from the document (and the sentence from which the side effect has been observed). In accordance with the exemplary embodiment, “explainable AI” is thus implemented.

The description of the sentence leading to the determination that the medicine has a side effect means that there is a description related to the side effect in the document. “Determining the side effect from the document” means a determination as to whether there is a description of the side effect in the document.

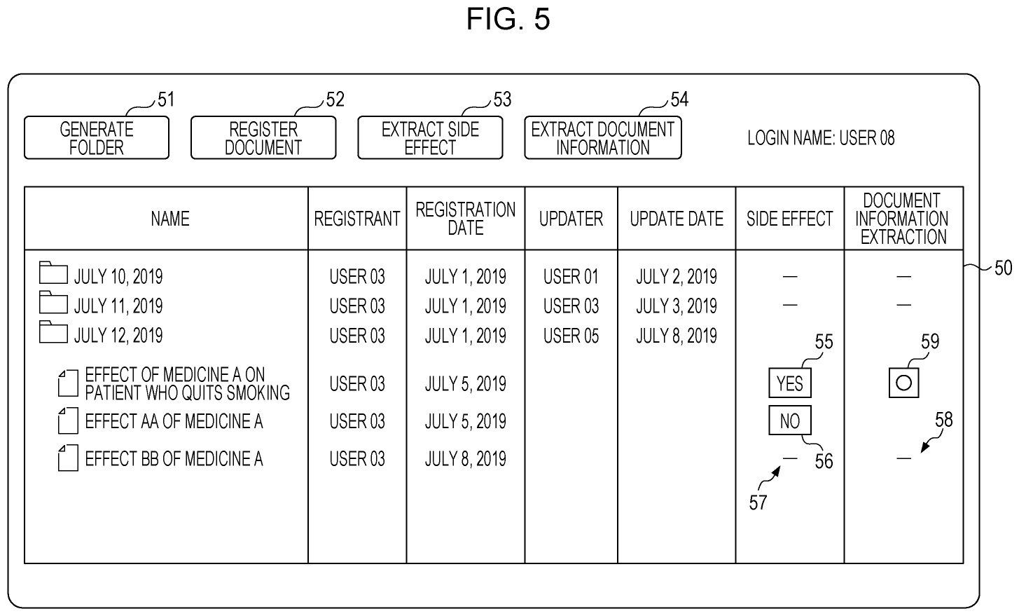

A user interface of the exemplary embodiment is described below. Illustrated images in FIGS. 5 through 11 are examples of displayed images. The information item values of the structured information displayed in the images are not necessarily consistent with the information item values included in the structured table in FIG. 4.

FIG. 5 illustrates a display example of a standard screen provided by the information processing apparatus 1 of the exemplary embodiment. If a predetermined operation is performed by the user who wishes to determine the side effect of a medicine described in the document, the UI processing controller 11 performs control to display the standard screen in FIG. 5 on a display in response to the user operation. The standard screen includes a document that may be specified as a process target, information on the document, and a display region 50 that displays the determination results of the side-effect determination process performed on the document. The display region 50 includes columns that lists display name, registrant, registration date, updater, update date, side effect, and document information extraction. The name column lists the name of a folder (folder name) storing a file that is a target of the process and registered in advance in response to a past user operation or the name of a document (document name) that is a target of the process and registered in advance in response to the past user operation. The registrant column lists information identifying a user who has registered the folder or document (such as a user name). The registration date column lists information indicating time and date on which the folder or document has been registered. The updater column lists information identifying a user who has updated the document stored in the registered folder (such as a user name). The update date column lists information identifying time and date on which the document has been updated. The information described heretofore is automatically set and displayed when the folder or document is registered in the name column. The side-effect and document information extraction columns are described below.

Referring to FIG. 5, a folder generation button 51, document registration button 52, side-effect extraction button 53, and information extraction button 54 are displayed above the display region 50. The process triggered by the selection of each of these buttons 51 through 54 is described below.

When the folder generation button 51 is selected by the user, the UI processing controller 11 causes an input screen to be displayed on a display. The input screen indicates the location of a folder where the folder is created and the folder name. If the folder is created with the user having entered predetermined items on the input screen, the UI processing controller 11 additionally displays the folder name of the created folder at the name column.

When the document registration button 52 is selected by the user, the UI processing controller 11 causes the input screen to be displayed. The input screen is used to receive a folder serving as a registration destination and a document name that is newly registered in the folder. When the user specifies predetermined items to be entered on the input screen, the UI processing controller 11 causes the specified document in the specified folder to be displayed at the name column. The user interface may set the user free from entering the folder name and document name and then may display a list of existing folders and documents such that the user may select ones from the list.

A menu screen (not illustrated) displaying a delete, copy, move, and change-name buttons is prepared in advance. The UI processing controller 11 causes the menu screen when the folder name or document name displayed in the name column is right-clicked by the user and performs the process corresponding to the button on the menu screen selected by the user.

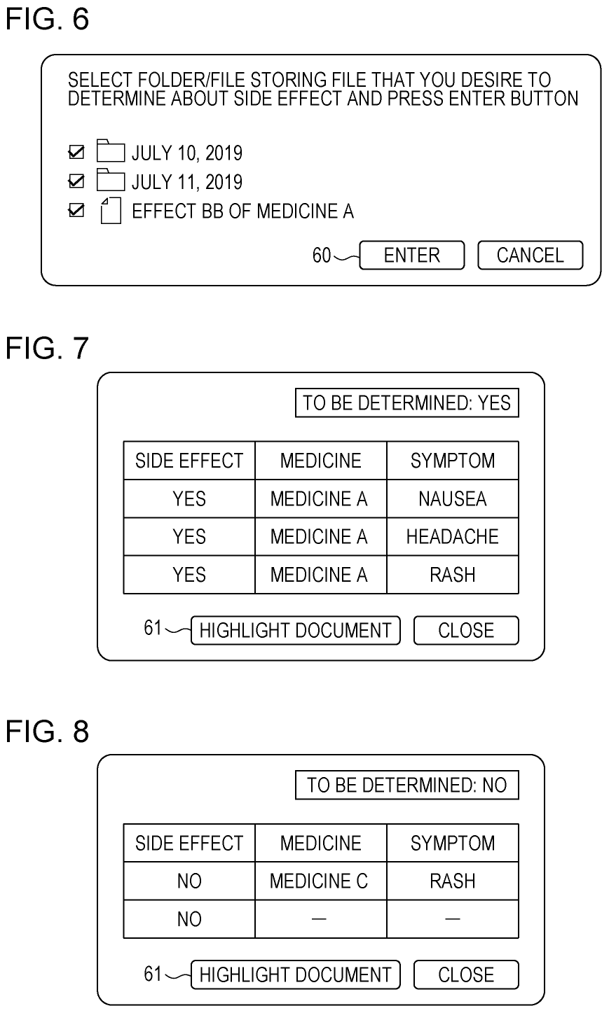

FIG. 6 illustrates a display example of a document selection screen displayed when the side-effect extraction button 53 is selected on the standard screen of the exemplary embodiment. If the side-effect extraction button 53 is selected by the user, the UI processing controller 11 extracts the folder storing only the document that has not undergone the side-effect determination and the document, and display the list on the document selection screen.

The user selects from the list a folder or document that serves as a side-effect determination target. Referring to FIG. 6, the user may select multiple folders or documents. The selection of a folder means the selection of all the documents (excluding a document has undergone the determination) included in the folder. If the user has selected an enter button 60 after selecting a folder or document, the side-effect determination process is performed.

The AI determination results are obtained through the side-effect determination process. The UI processing controller 11 causes the determination results to be displayed on the standard screen. Referring to FIG. 5, if a side effect is determined to be present, a side-effect present button 55 is displayed in association with the document serving as the determination target, and if no side effect is determined to be present, a side-effect absent button 56 is displayed in association with the document serving as the determination target. A dash 57 indicates that the corresponding document is not yet selected as the side-effect determination target.

By referring to item columns indicating the presence or absence of a side effect, the user recognizes that the AI determination has been performed and the determination results. To retrieve further details of the determination results, the user may select the button 55 or 56.

FIG. 7 illustrates a display example of a side-effect sub-screen displayed when the side-effect present button 55 is selected on the standard screen of the exemplary embodiment. FIG. 8 illustrates a display example of a side-effect sub-screen displayed when the side-effect absent button 56 is selected on the standard screen of the exemplary embodiment. Referring to FIG. 7, the UI processing controller 11 extracts the structured information of the sentence that is determined to be side-effect present from the structured information of the document corresponding to the side-effect present button 55 on the structured-information memory 18. The UI processing controller 11 then causes the extracted structured information to be displayed on the side-effect sub-screen. Referring to FIG. 8, the UI processing controller 11 extracts the structured information of the sentence that is determined to be side-effect absent from the structured information of the document corresponding to the side-effect absent button 56 on the structured-information memory 18. The UI processing controller 11 then causes the extracted structured information to be displayed on the side-effect sub-screen.

Referring to FIGS. 7 and 8, a side-effect column lists the side-effect AI determination of each sentence. A medicine column lists a medicine name included in the structured information, namely, a medicine name of a medicine serving as a side-effect determination target. A symptom column lists a symptom included in the structured information.

In accordance with the exemplary embodiment, information items are extracted from the change information and are displayed on the side-effect sub-screen. The information items to be extracted are not limited to these items. Instead of extracting these items, all information items included in the structured table may be displayed. The determination results may be displayed in a mixed form without separating the side-effect present determination in FIG. 7 from the side-effect absent determination in FIG. 8.

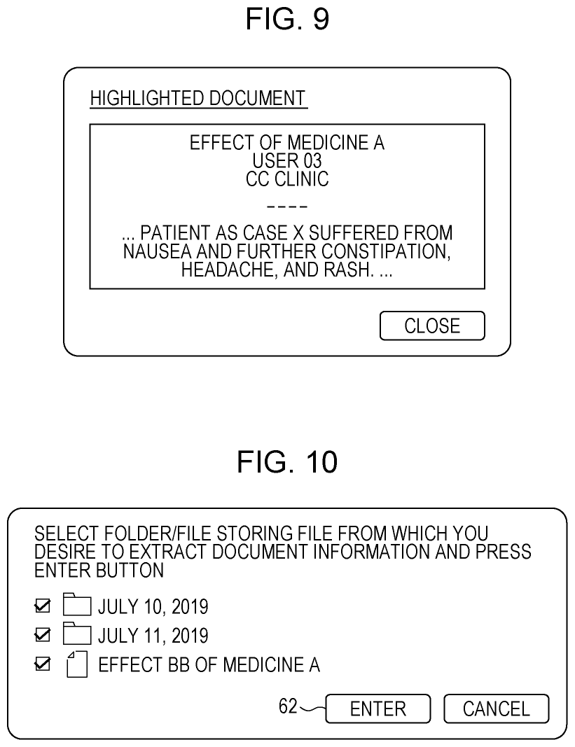

FIG. 9 illustrates a display example of a document display screen displayed when a document highlight button 61 is selected on the side-effect sub-screen of the exemplary embodiment. If the document highlight button 61 is selected by the user, the UI processing controller 11 causes the document information retrieval unit 12 to retrieve a document serving as a process target. The UI processing controller 11 then causes the retrieved document to be displayed on the document display screen. The UI processing controller 11 then highlights corresponding words in the document. The corresponding words are those corresponding to the medicine and symptom displayed on the side-effect sub-screen. The exemplary embodiment is not limited to this example. For example, the corresponding words may be all the words included in the structured information. Referring to FIG. 9, the corresponding words are highlighted such that they are discriminated from the remaining portion of the document. The display form other than the highlighting may be employed. For example, the color of the characters may be changed such that the corresponding words are easy to look at in the displayed document.

FIG. 10 illustrates a document selection screen displayed when the document information extraction button 54 is selected on the standard screen of the exemplary embodiment. If the document information extraction button 54 is selected by the user, the UI processing controller 11 extracts a folder, storing only a document for which document information has not yet been produced, and the document. The dash 58 in the document information extraction column on the standard screen in FIG. 5 indicates that the document information has not yet produced for the document. Specifically, the document (and the name of the folder including the document) corresponding to the dash 58 is displayed in a list on the document selection screen.

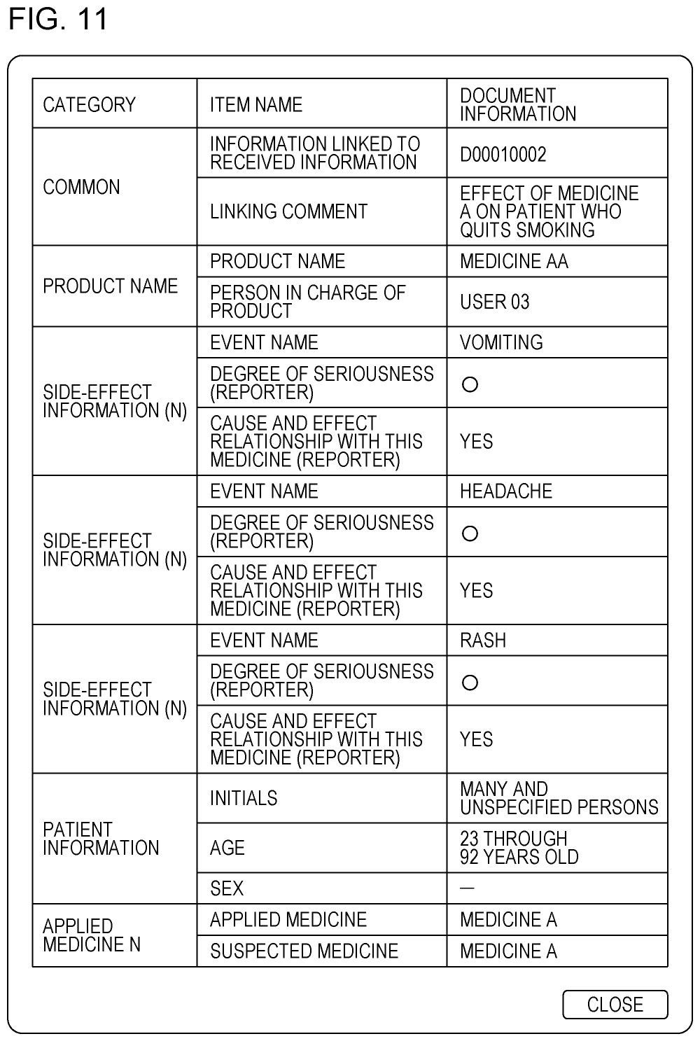

If an enter button 62 is selected after the folder or the document is selected by the user, the document information retrieval unit 12 receives the selected document name (or folder name) from the UI processing controller 11. The document information retrieval unit 12 extracts information for each column illustrated in FIG. 11 by analyzing the document and stores the information as document information on the document information memory 19. In response to an instruction from the document information retrieval unit 12, the UI processing controller 11 causes a document information display button 59 to be displayed at the location corresponding to the document as the process target at the document information extraction column on the standard screen.

The user now recognizes the production of the document information by the displayed document information display button 59. If the document information display button 59 is selected by the user, the UI processing controller 11 acquires via the document information retrieval unit 12 the document information of the document corresponding to the selected document information display button 59 and causes the document information to be displayed on the document information display screen. FIG. 11 illustrates an example of the document information display screen displayed when the document information display button 59 is selected.

In accordance with the exemplary embodiment, the information related to the document that is determined to be side-effect present is thus displayed.

The foregoing description of the exemplary embodiment of the present disclosure has been provided for the purposes of illustration and description. It is not intended to be exhaustive or to limit the disclosure to the precise forms disclosed. Obviously, many modifications and variations will be apparent to practitioners skilled in the art. The embodiment was chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. An information processing apparatus, comprising:
an extraction unit that extracts, from each sentence in a document, medicine information identifying a medicine, symptom information related to a symptom, and change information representing a change in a condition of a patient; and
a generation unit that, after the medicine is taken by the patient, generates structured information that is used to determine a side effect from the information extracted by the extraction unit.

2. The information processing apparatus according to claim 1, wherein by analyzing the document, the extraction unit extracts, together with the information, information as to whether the medicine has been taken, information as to whether the symptom has been observed, and information as to whether the change has occurred.

3. The information processing apparatus according to claim 2, further comprising a determination unit that, by referring to the structured information, determines the side effect of the medicine.

4. The information processing apparatus according to claim 3, wherein the determination unit determines the side effect on a per sentence basis.

5. The information processing apparatus according to claim 3, wherein the determination unit determines the side effect from a plurality of sentences or the document.

6. The information processing apparatus according to claim 3, wherein the determination unit determines seriousness of the side effect.

7. The information processing apparatus according to claim 3, wherein the determination unit determines newness of the side effect.

8. The information processing apparatus according to claim 3, wherein the determination unit determines the side effect in accordance with a fact that the symptom has been observed in response to use of the medicine.

9. The information processing apparatus according to claim 3, further comprising a display controller that performs control to display determination results obtained by the determination unit.

10. The information processing apparatus according to claim 9, wherein the display controller performs control to display in an associated form the structured information of each of the sentences and the determination results obtained from the sentence by the determination unit.

11. The information processing apparatus according to claim 9, wherein the display controller performs control to display the medicine information, the symptom information, and the change information of the sentence in accordance with which the determination unit determines that the side effect has been observed.

12. The information processing apparatus according to claim 9, wherein the display controller performs control to display determination results of the side effect with reference to the document, the determination results being obtained from the structured information of each of the sentences by the determination unit.

13. An information processing apparatus, comprising a determination unit that, using a pre-generated learning model, determines a side effect from structured information after a medicine is taken by a patient, wherein the structured information is extracted and generated from each sentence in a document and includes medicine information identifying the medicine, symptom information related to a symptom, and change information representing a change in a condition of the patient.

14. The information processing apparatus according to claim 13, wherein the determination unit determines the side effect on a per sentence basis.

15. The information processing apparatus according to claim 13, wherein the determination unit determines the side effect from a plurality of sentences or the document.

16. The information processing apparatus according to claim 13, wherein the determination unit determines seriousness of the side effect.

17. The information processing apparatus according to claim 13, wherein the determination unit determines newness of the side effect.

18. The information processing apparatus according to claim 13, wherein the determination unit determines the side effect in accordance with a fact that the symptom has been observed in response to use of the medicine.

19. A non-transitory computer readable medium storing a program causing a computer to execute a process for processing information, the process comprising:
extracting, from each sentence in a document, medicine information identifying a medicine, symptom information related to a symptom, and change information representing a change in a condition of a patient; and
after the medicine is taken by the patient, generating structured information that is used to determine a side effect from the extracted information.

20. A non-transitory computer readable medium storing a program causing a computer to execute a process for processing information, the process comprising, with a pre-generated learning model, determining a side effect from structured information after a medicine has been taken by a patient, wherein the structured information is extracted and generated from each sentence in a document and includes medicine information identifying the medicine, symptom information related to a symptom, and change information representing a change in a condition of the patient.
